(19) United States
(12) Patent Application Publication
(10) Pub. No.: US 2024/0058530 A1
(43) Pub. Date: Feb. 22, 2024

(54) AUTOINJECTOR WITH A PUMP

(71) Applicant: Sanofi, PARIS (FR)

(72) Inventors: Ramsay Black, Melbourn, Hertfordshire (GB); Uwe Dasbach, Frankfurt am Main (DE); Thomas Mark Kemp, Melbourn, Hertfordshire (GB); William Timmis, Melbourn, Hertfordshire (GB)

(21) Appl. No.: 18/267,044

(22) PCT Filed: Dec. 16, 2021
(86) PCT No.: PCT/EP2021/086104

§ 371 Date: Jun. 13, 2023

(30) Foreign Application Priority Data
Dec. 17, 2020 (EP) 20315494.3

(51) Int. Cl. A61M 5/145; A61M 5/20

(52) U.S. Cl. CPC A61M 5/14586; A61M 5/20

(57) ABSTRACT

The invention relates to an autoinjector comprising —a housing (105) which is configured to receive an operating reservoir (120, 220) with a fluid, —a pump arrangement (100, 200), which is configured to drive the fluid from the operating reservoir (120, 220) towards and through an outlet (180) of the autoinjector in a dispensing operation.

### FIELD OF INVENTION

The present disclosure relates to an autoinjector with a pump.

### BACKGROUND OF INVENTION

In regular drug delivery devices, where a single drive mechanism which may be housed in a housing of the drug delivery device is used in conjunction with several cartridges or ampules to dispense drug contained in the cartridge or ampule from the device, usually a cartridge holder of the device is releasably connected to the housing and can be removed from the housing to replace a used cartridge.

Devices of this kind, however, are designed for housing a cartridge and a syringe, and due to the shape of these components the overall shape of the device is adapted to the syringe and the cartridge, which is usually decisive for the form factor of the device.

### SUMMARY

It is an object of the present disclosure to provide an alternative autoinjector. This object is solved by the present disclosure and, particularly, by the subject-matter of the independent claim. Advantageous embodiments and refinements are subject to the dependent claims.

The disclosure relates to an autoinjector comprising a housing, which is configured to receive an operating reservoir with a fluid, e.g. a liquid, and a pump arrangement, which is configured to drive the fluid from the operating reservoir towards and through an outlet of the autoinjector in a dispensing operation.

Using a pump for driving the fluid from the operating reservoir to the outlet has the advantage that the amount of fluid to be moved and delivered through the outlet can be controlled by the pump. Also there is some degree of freedom to choose the shape of the reservoir. For example it can have a different shape than a syringe.

In an embodiment the operating reservoir comprises a tube, a pipe or another hollow shaped body which is configured for moving fluid through it.

In an embodiment, the operating reservoir comprises a flexible material, such as a flexible plastic, preferably an elastomer. The operating reservoir may be elastically deformable. That is to say, when deformed, the operating reservoir tends to resume its undeformed shape. This may be particularly suitable when the pump arrangement involves deforming the operating reservoir during its operation, as for example in a peristaltic pump arrangement. The pouch may comprise more than one material which may be specific adaptable to its inside and outside according to chemical and/or mechanical requirements. For example regarding the outside of the pouch the material may be mechanically robust with regard to the pressure applied to the pouch by squeezing. Regarding the inside of the pouch a material may be required which does not engage in a chemical reaction with the fluid contained in the pouch.

In an embodiment the outlet comprises a needle which may be used for dispensing a medicament to a patient.

In an embodiment the pump arrangement comprises a movable element which is operatively connected with the operating reservoir such that a movement of the movable element drives at least a portion of the fluid from the operating reservoir to the needle. The movable element may be configured to undertake a repeating, preferably periodically repeating, movement, such that with every repetition a portion of the fluid is moved from the operating reservoir to the needle. The total amount of fluid which is moved from the operating reservoir to the needle can then be determined by the number of repetitions.

In an embodiment the autoinjector comprises a motor spring which is configured to move the movable element. The motor spring may be biased when it is assembled along with the movable element. The motor spring may be activated with a trigger so its stored potential energy translates into kinetic energy, thereby driving the movable element. The movable element in turn drives the fluid from the operating reservoir. Depending on the configuration of the motor spring with respect to the stored energy the overall distance of the movement can be determined. By predetermining the distance of movement of the movable element the amount of fluid which is driven through the operating reservoir and through the needle can be indirectly predetermined. In case the fluid relates to a medicament, the dose of the medicament can be adjusted, for example by the size of the reservoir or the number of revolutions by the movable element which correlates to the design of the spring. These adjustments can be done in the process of assembling the device. Once the device is assembled the amount of dose will be released which is provided by the configuration.

In an embodiment the autoinjector comprises a reserve reservoir which is in fluid communication with the operating reservoir, and which is configured to supplement fluid to the operating reservoir during a dispensing operation. This keeps available a larger amount of fluid which can be provided to the outlet for a dispensing operation.

The operating reservoir expediently has a smaller diameter than the reserve reservoir. When the fluid is driven through the operating reservoir the fluid of the reserve reservoir is directly substituting the removed amount of fluid from the operating reservoir. The larger amount of fluid and the larger diameter of the reserve reservoir lead to a fluid pressure from the reserve reservoir to the operating reservoir. A continuous flow of fluid can be established from the reserve reservoir to the operating reservoir as soon as fluid is moved out of the operating reservoir. This is of importance when a dispension of a medicament is undertaken where a continuous flow of liquid is essential, and at the same time a predetermined dose needs to be ensured.

In an embodiment the reserve reservoir comprises a flexible primary pack which may be a pouch. Employing a flexible primary pack has the advantage of improved robustness, in particular compared to a glass syringe which is fragile and can break. Further, there is an Improved drug integrity and less contamination risk, because the reservoir only comprises one opening, which needs to be sealed, which is on the injection side. In autoinjectors with syringes there is additionally the side of the stopper to be sealed.

Another advantage is the opportunity for a different form with usability benefits, because the reservoir can be adjusted to the form of the device. Its compactness and consistency avoids large PFS tolerances reducing injection variability. In particular it is possible to employ plastic instead of glass for the reservoir which can be manufactured with a higher precision compared to glass. It further has a reduced stalling risk and no stopper friction, because there is no stopper required. The reservoir can be filled by vacuum filling to eliminate any air, or steam purging prior to container closure. In the case of vacuum filling the pouch can be pulled apart (e.g. by a vacuum) which creates a vacuum inside the pouch. This pulls liquid from a connected container inside the pouch.

In an embodiment, the reserve reservoir may be flexible, particularly collapsible. A collapsible reservoir may have an inner cross-section which decreases as fluid is drained or removed from that reservoir. When the reservoir has been emptied formerly spaced inner surfaces defining the diameter of the reservoir may contact one another.

In an embodiment, aside from the fluid connection to the outlet of the autoinjector, and a fluid interconnection with each other, the operation reservoir and the reserve reservoir may be closed with respect to the exterior.

In an embodiment the autoinjector comprises a circumferentially disposed element which encloses a rotation axis, which is arranged adjacent to the reserve reservoir radially inward. This ensures a stable arrangement of the reserve reservoir and provides packaging efficiency.

In an embodiment the movable element comprises a squeeze unit with at least one squeeze element which is configured to squeeze the operating reservoir, wherein the squeeze unit is rotatable around the rotation axis, such that the at least one squeeze element rotates around the rotation axis and thereby squeezes the operating reservoir causing the fluid to be driven within the operating reservoir. It is advantageous to squeeze the operating reservoir rather than the fluid itself. In this configuration the fluid is moved by an indirect movement of the squeeze element towards the operating reservoir. The fluid can be maintained in a closed loop and it is only released when it exits the needle. The rotation of the squeeze element further has the advantage that a repeatable squeezing of the operating reservoir, and thereby movement of the fluid, is possible.

The squeeze element may comprise or may be a roller. The squeeze element may be configured to rotate around a further rotation axis while at the same time rotating around the rotation axis. The further rotation axis may run through the squeeze element. The rotation axis may be outside of the squeeze element. The further rotation axis may run parallel to the rotation axis or may form an acute angle with the rotation axis, e.g. of less than 10°.

The squeeze unit may comprise an arm which extends or is orientated in radial direction. The arm may have a first end closer to the rotation axis and a second end located radially outwardly with respect to the first end, i.e. more distant to the rotation axis. The squeeze element may be arranged at one end of the arm, e.g. at the second end of the arm.

The squeeze unit may comprise two or more, e.g. three, such arms. Each of the arms may be assigned a squeeze element. The arms may be arranged symmetrically around the rotation axis.

The squeeze unit may be arranged such that, when the squeeze element squeezes the operating reservoir, the squeeze element presses against the operating reservoir in radial outward direction. In other word, when the operating reservoir is squeezed, it is compressed in radial direction and/or expanded in axial direction.

In an embodiment the operating reservoir comprises a flexible material, so that it can be squeezed effectively. The rotation of the squeeze element around the rotation axis ensures a related squeezing of the operating reservoir.

The pump arrangement, including the operating reservoir with the squeezing element, may comprise a peristaltic pump.

In an embodiment the operating reservoir is arranged circumferentially around the rotation axis. In this way the squeeze element, which is rotatable around the rotation axis, may continuously squeeze the operating reservoir and thereby causing a continuous flow of fluid through the needle. The operating reservoir may be arranged circumferentially around the rotation axis and comprises a cross-section of a circle segment. In such a configuration the reserve reservoir may be arranged radially outward with respect to the operating reservoir. The reserve reservoir may also be arranged circumferentially around the rotation axis. The reserve reservoir may also comprise a cross-section of a circle segment whereas one end of the reserve reservoir is in fluid communication with the operating reservoir, thereby connecting the larger and radially more outward arranged reserve reservoir with the operating reservoir, which is radially inward placed and comprising a smaller radius of circle segment. The reserve reservoir and the operating reservoir comprise a spiral-shaped arrangement. The open ending of the operating reservoir is connected to the needle.

In an embodiment the circumferentially disposed element is radially arranged between the operating reservoir and the reserve reservoir. The circumferentially disposed element may be in mechanical contact with the operating reservoir and/or the reserve reservoir. For example, the circumferentially disposed element mechanically supports the operating reservoir, e.g. while it is squeezed by the at least one squeeze element in radial direction.

During the rotation of the squeeze element the operating reservoir is squeezed, and thereby pushed radially outward. The circumferentially disposed element is arranged radially outward and, preferably, adjacent to the operating reservoir. The radially outward pushed operating reservoir may be mechanically supported by the circumferentially disposed element. This not only ensures that a portion of the operating reservoir is moved outward, but it also ensures that the force of the squeeze element towards the operating reservoir is reliably transferred into squeezing and in consequence in moving the fluid through the operating reservoir.

In an embodiment the circumferentially disposed element comprises a cylindrical wall. A cylindrical shape provides an even surface in the direction around the rotation axis. The cylindrical wall may have a cross-section of a circle segment.

In an embodiment the at least one squeeze element comprises a roller which is configured to squeeze the operating reservoir. The roller may be arranged at the radial outward ending of the squeeze unit, so that it is in direct contact with the operating reservoir for squeezing the operating reservoir. When the squeeze unit rotates around the rotation axis the roller squeezes the operating reservoir. The roller itself rotates around its own rotation axis which is radially offset from the rotation axis of the squeeze unit and extends through the roller. The roller may rotate in the opposite rotation direction as the squeeze unit. The rolling function of the roller leads to a reduced resistance caused by friction between roller and operating reservoir while the roller is moved around the rotation axis of the squeeze unit together with the rotating squeeze unit.

In an embodiment the squeeze unit comprises at least two squeeze elements. The squeeze elements are configured to squeeze the operating reservoir simultaneously, preferably at angularly offset locations. This ensures that a continuous movement of fluid through the operating reservoir is maintained.

In an embodiment the squeeze unit comprises three squeeze elements which are arranged at a circular path around the rotation axis. The three squeeze elements may be arranged along a circle with equal spacing's between each other. Preferably each two squeeze elements are separated by an angle of 120°. It is also possible that the three separations between the squeeze elements are 90°, 130° and 70°. It is also possible that there are more than three squeeze elements.

In an embodiment the motor spring is mechanically connected to the squeeze unit and provides rotation energy to the squeeze unit to drive the squeeze unit in the direction of rotation. The motor spring may be biased when it is assembled along with the movable element. The motor spring may be activated with a trigger so its stored potential energy translates into kinetic energy, thereby driving the squeeze unit. The squeeze unit and the squeeze element(s) in turn drive the fluid from the operating reservoir. Depending on the configuration of the motor spring with respect to the stored energy it can be determined the overall distance of the movement.

By predetermining the distance of movement of the squeeze element the amount of fluid which is driven through the operating reservoir and through the needle can be indirectly predetermined. In case the fluid relates to a medicament, the dose of the medicament can be adjusted, for example by the size of the reservoir or the number of revolutions by the movable element which correlates to the design of the spring. These adjustments can be done in the process of assembling the device. Once the device is assembled the dose is set.

In an embodiment the autoinjector comprises a rotary gear system which is mechanically connected to the squeeze unit, and which is configured to cause the squeeze unit to rotate around the rotation axis, wherein the speed of rotation of the squeeze unit is determined by a gear ratio of the rotary gear system. The gear ratio of the rotary gear system can be adjusted such that the force which is required to turn rotary gear system which drives the squeeze unit and thereby compensates the resistance of the operating reservoir and the fluid can be reduced. This is of advantage in cases where the squeeze unit is turned with an energy source, which has only stored a limited amount of energy in order to drive the squeeze unit. Such an energy source can be a motor spring which is mechanically connected to the rotary gear system and provides rotation energy to the rotary gear system.

In an embodiment the rotary gear system may comprises a planetary gear system, which comprises a shaft which rotates around the rotation axis.

In an embodiment the movable element comprises a diaphragm actuator, which is integrated into a diaphragm pump which is in fluid communication with the operating reservoir, wherein the diaphragm actuator is configured to move axially, e.g. parallel to the rotation axis, such that the movement causes at least a portion of the fluid to flow from the operating reservoir through the diaphragm pump to the needle. A housing of the diaphragm pump may be stationary arranged, whereas the corresponding diaphragm actuator and the diaphragm are moving. According to the principle of a diaphragm pump the movement of the diaphragm is oblique, preferably perpendicular, to the direction of movement of the fluid within the housing of the pump. Therefore, the diaphragm pump requires only little space during operation.

In an embodiment the circumferentially disposed element which is rotatable around the rotation axis, comprises a mechanical guiding feature, wherein the diaphragm actuator is connected to the mechanical guiding feature such that when the circumferentially disposed element rotates around the rotation axis the diaphragm actuator is moved parallel to the rotation axis, thereby causing the fluid to flow towards the needle.

The movement of the diaphragm actuator may be an oscillating movement, such that a movement in one direction parallel to the rotation axis is followed by a movement in the opposite direction parallel to the rotation axis.

Using a diaphragm pump is advantageous because no external load is applied to the operating reservoir. The flow of the fluid occurs solely through the movement of the diaphragm actuator, which pulls and pushes the diaphragm leading to a movement of the fluid through the operating reservoir. Also, this configuration does not require a separate gearing system. This pump arrangement has a lower torque requirement and a higher pump efficiency compared to a peristaltic pump arrangement, and it facilitates a smaller device size.

In an embodiment the mechanical guiding feature comprises a sinusoidal circumferential guiding feature. This ensures a periodical movement of the diaphragm actuator and/or providing a continuous flow of fluid through the operating reservoir.

When the circumferentially disposed element rotates clockwise, the sinusoidal form of the guiding feature may cause the diaphragm actuator to move in an upward direction according to the wave. This draws fluid from the operating reservoir, filling the diaphragm. When the circumferentially disposed element continues to rotate the sinusoidal form causes the diaphragm actuator to move downwards at some point, pushing the drug out of the pump. As the circumferentially disposed element continues to rotate, the diaphragm actuator moves upwards again, refilling the pump. Two axial end surfaces of the guiding feature which face in different axial directions may guide the movement of the actuator during operation of the device. One axial end surface may guide the movement in the upward direction, i.e. away from the fluid or the housing, and the other one may guide the movement in the downward direction.

The circumferentially disposed element may comprise a cylindrical cam which is rotatable around a rotation axis.

In an embodiment the mechanical guiding feature is arranged on the radial inside or on the radial outside of the mechanical guiding feature. This supports an integrated device, because the diaphragm pump is then also arranged radially inside the cylindrical cam.

In an embodiment the mechanical guiding feature comprises a groove or a ridge. A groove has the advantage that a mechanical connection with the diaphragm actuator can be established with a simple pin which engages into the groove. A ridge has the advantage that a mechanical connection needs to be fixed from two sides of the ridge which is possibly more stable than a single pin in a groove.

In an embodiment the operating reservoir and the reserve reservoir are arranged in a common plane perpendicular to the rotation axis, if applicable, aside from an end portion of the operating reservoir close to the outlet, where the end portion may be connected to the needle.

In an embodiment the autoinjector comprises an outlet drive mechanism comprising
an outlet,
an interface element which is connected to or integrated with the outlet, wherein the interface element is movable from a first axial position to a second axial position along the rotation axis,
a trigger which is operatively connected to the interface element wherein the trigger is movable from a first trigger position to a second trigger position along the rotation axis, wherein
   in the first trigger position the interface element is releasably locked from moving from the first axial position to the second axial position, and
   in the second trigger position, the interface element is movable to the second axial position, wherein
   the movement of the trigger from the first trigger position to the second trigger position causes the interface element to be released from the first axial position such that the interface element is movable to the second axial position.

The outlet may comprises a needle. The interface element may comprise at least one interface feature. The interface element may comprise a needle holder. The interface feature may comprise a needle holder surface which is arranged at the needle holder and which is oriented rectangular with respect to the rotation axis. Preferably the needle holder comprises a needle holder ledge, wherein the needle holder surface is arranged at the needle holder ledge. The needle holder may comprise two needle holder ledges which are arranged opposite to each other with respect to the rotation axis, and each comprising a needle holder surface. The needle holder may comprise a cylindrical body wherein its axis is the rotation axis. The two needle holder ledges are arranged on the radial outside of the cylindrical body of the needle holder. The needle holder may comprise mechanical guides, for example axial grooves, on the radial outside of the cylindrical body for guiding a movement of trigger along the rotation axis.

The trigger may comprise a button, one ore more trigger arms and one or more trigger interfaces. The trigger arms may extend from the button. Preferably, the trigger comprises two trigger arms. The trigger interfaces are arranged at the endings of the trigger arms. The trigger interfaces may comprise oblique trigger surfaces.

When the trigger moves along the rotation axis the trigger arms may be guided by the mechanical guides of the needle holder, wherein the mechanical guides of the needle holder secure the trigger arms against rotation. It is also possible that the trigger arms are secured against rotation by a base element which comprises mechanical guidance and which is fixed to or integrated with the housing of the device. The base element may comprise a base element main body which is fixed to or integrated with the housing. The base element main body may comprise a hole which may be arranged on the rotation axis and which is configured so that the needle is movable through the hole along the rotation axis. The hole is may be sealed by a sealing which seals hole and the housing towards the outside. The sealing may be penetrated by the needle when the needle is moved by the drive spring in the direction of the hole. The base element may comprise base element arms which extend from the base element main body towards the inside of the housing of the device. The base element arms are arranged around the rotation axis and are spaced apart with a spacing. The spacings between the base element arms are configured to receive the radial outward part of the trigger arms for guiding them axially in the assembled state. In the assembled state the needle holder ledges are received in the other spacings between the base element arms and are secured against rotation with respect to the base element and because the base element is fixed to or integrated with the housing the needle holder ledges and the needle holder is secured against rotation with respect to the housing.

The outlet drive mechanism is enables the release of the needle to move to a position for injection when a trigger is moved from a first trigger position to a second trigger position. In order to avoid an accidental movement of the needle the needle holder is locked to a first axial position until the trigger is moved to its second trigger position.

In an embodiment the autoinjector comprises a holding element which is in mechanical contact with the interface element, wherein
the holding element is rotatable around the rotation axis relative to the interface element from a blocking position to a release position, wherein
in the blocking position the interface element is releasably locked by the holding element to move from the first axial position to the second axial position, and
in the release position the interface element is movable from the first axial position to the second axial position, wherein
the movement of the trigger from the first trigger position to the second trigger position causes the holding element to rotate from the blocking position to the release position.

The holding element may comprise at least one first holding interface which is configured to interact with one of the trigger surfaces. The holding element may comprise a collar. The collar comprises one ore more collar trigger arms, preferably two trigger arms, which are arranged opposite to each other with respect to the rotation axis. The one or more collar trigger arms comprise first holding interfaces. The first holding interfaces may comprise first holding surfaces which may be oblique holding surfaces, and which face the trigger interfaces which may be oblique trigger surfaces along the rotation axis. The oblique trigger surfaces are configured to interact with the oblique holding surfaces. The oblique trigger surfaces and the oblique holding surfaces are configured and oriented to slide on top of each other. When the trigger is moved from the first trigger position to the second trigger position the oblique trigger surfaces and the oblique holding surfaces get in mechanical contact such that the oblique trigger surfaces are pushed on the oblique holding surfaces. When the surfaces slide on top of each other the axial movement of the trigger arms and the oblique trigger surfaces, which apply axial forces to the oblique holding surfaces and the collar trigger arms, causes the collar trigger arms and the collar to rotate around the rotation axis. The rotation of the collar is relative to the needle holder and the needle holder ledges which are secured against rotation.

It is also possible that only one of the trigger surfaces and the holding surfaces is oblique.

The holding element may further comprise at least one second holding interface which is configured to interact with the interface element, in particular with the interface feature, which may be a surface of the needle holder ledge. The holding element may comprise one or more collar holding arms, preferably two holding arms, which are arranged opposite to each other with respect to the rotation axis. Each of the collar holding arms comprise a second holding interface which may comprise a second holding surface. Each of the second holding surfaces face an interface feature which may be needle holder surface. In the first axial position of the needle holder the needle holder surfaces are in mechanical contact with the second holding surfaces such that when the collar holding arms rotate relative to the needle holder the needle holder surfaces slide on top of the second holding surfaces. Thereby the second holding surfaces block the needle holder surfaces from moving in the axial direction.

The one ore more collar trigger arms and the one or more collar holding arms are arranged alternating around the rotation axis, wherein each neighbouring collar trigger arm and collar holding arm are separated by a spacing. The spacings are configured to receive the needle holder ledges and/or the trigger arms.

In an assembled state of the outlet drive mechanism the collar is arranged inside the volume which is enclosed by the base element arms, such that the collar can rotate inside the base element.

When the trigger is moved from the first trigger position to the second trigger position the applied force to the collar trigger arms causes the collar trigger arms to rotate relative to the needle holder and the needle holder ledges. The rotation continues until the needle holder ledges face the spacings of the collar.

Further the needle holder ledges are blocked by the collar trigger arms against axial movement by the collar holding arms in the first axial position of the needle holder. Further, in the release position of the holding element the drive spring moves the needle holder to the second axial position. The blocking and release position of the collar are only different in rotation/angle but not in the axial position.

The trigger interface is further configured to translate an axial movement of the trigger and its interface into a rotational movement a holding element. The trigger interface may comprise arms which extend along the rotation axis, such that they can mechanically interact with the collar.

The collar provides an additional safety with regard to an accidental movement of the needle. The needle holder is only released when the collar has undertaken a rotational movement which is dependent to the axial movement of the trigger. The needle holder cannot rotate on its own because it is secured against rotation. Further in the blocking position of the collar the surfaces of the collar holding arms are pressed towards the surfaces of the needle holder ledges by the compressed drive spring. This ensures that the collar cannot rotate accidentally.

In an embodiment the autoinjector comprises an outlet drive unit which is operatively connected to the trigger and to the interface element, wherein
the outlet drive unit being configured to provide energy for moving the interface element from the first axial position to the second axial position, wherein the outlet drive unit has a first drive unit state and a second drive unit state, wherein
in the first drive unit state the outlet drive unit has energy stored and the interface element is in the first axial position and the holding element is in the blocking position and the interface element is prevented from moving to the second axial position, and
in the second drive unit state the outlet drive unit can transfer energy to the interface element so that the interface element is moved the first axial position to the second axial position along the rotation axis when the holding element is in the release position, wherein
the movement of the trigger from the first trigger position to the second trigger position causes the outlet drive unit to change from the first drive unit state to the second drive unit state.

The outlet drive unit may comprise a drive spring. The drive spring may be arranged between the trigger and the needle holder along the rotation axis. When the needle holder is in the first axial position, the drive spring is in the first drive unit state where the drive spring is compressed. When the needle holder is released so that it can move to the second axial position the drive unit is in the second drive unit state where the drive spring can expand and transfer mechanical energy to the needle holder. Under the force of the expanding drive spring the needle holder moves to its second axial position along the rotation axis.

When the release of the needle holder is initiated by the trigger the needle holder is then moved by the drive spring to the second axial position which can be the position for injection and dispension. In this way the trigger initiates an injection which occurs automatically and is driven by the drive spring once the trigger has initiated the release of the needle holder.

In an embodiment the trigger may also cause the dispension of the fluid.

In an embodiment the housing may has a shape with an overall base which has a larger diameter than the height, which extends along the longitudinal axis. In an embodiment the shape comprises a cylinder, in particular a cylinder with rounded edges.

In an embodiment the autoinjector is a disposable or single-use device, for providing a single dose.

### BRIEF DESCRIPTION OF THE DRAWINGS

Embodiments will now be described, by way of example only, with reference to the accompanying drawings, in which:

FIGS. 1A to 1F show schematic views of a peristaltic pump arrangement.

FIG. 1A shows a top view of a reduced peristaltic pump arrangement.

FIG. 1B shows an angled view of the peristaltic pump arrangement.

FIG. 1C shows an angled top-view of a peristaltic pump arrangement with a planetary gear.

FIG. 1C shows an angled bottom-view of a peristaltic pump arrangement with a planetary gear.

FIG. 1E shows an angled view of the planetary gear with a motor spring.

FIG. 1F shows cross-section of the peristaltic pump arrangement and a needle drive mechanism.

FIG. 2A shows an angled view of the diaphragm pump arrangement.

FIG. 2B shows cross-section of the peristaltic pump arrangement and a needle drive mechanism FIG. 2C shows an angled view of the diaphragm pump arrangement with a motor spring.

FIGS. 3A to 3C show a cut out of the diaphragm pump in different positions of operation.

FIG. 4A shows an exploded view of the needle drive mechanism.

FIG. 4B shows a view of the needle drive mechanism with the drive spring in a first state and assembled with a base element.

FIG. 4C shows a view of a needle drive mechanism with a drive spring in a first state.

FIG. 4D shows a view of the needle drive mechanism with the drive spring in a second state.

### DETAILED DESCRIPTION

The same reference numbers apply to the same features throughout the figures and the following explanations.

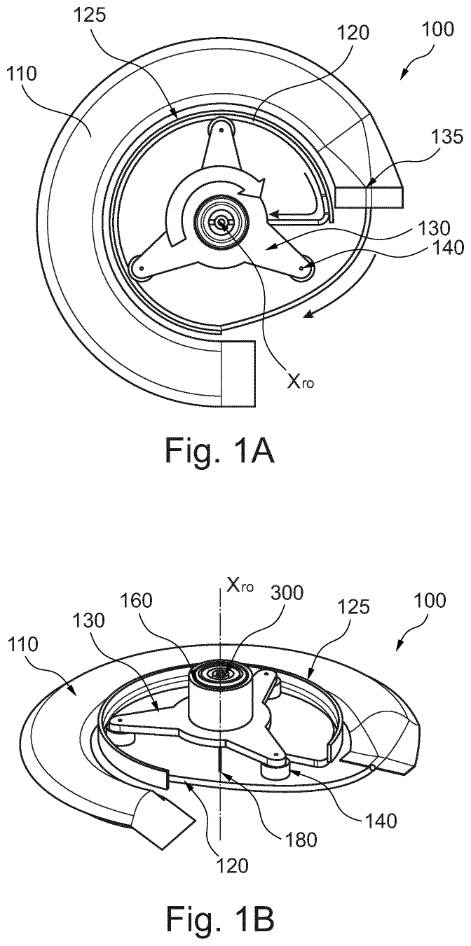

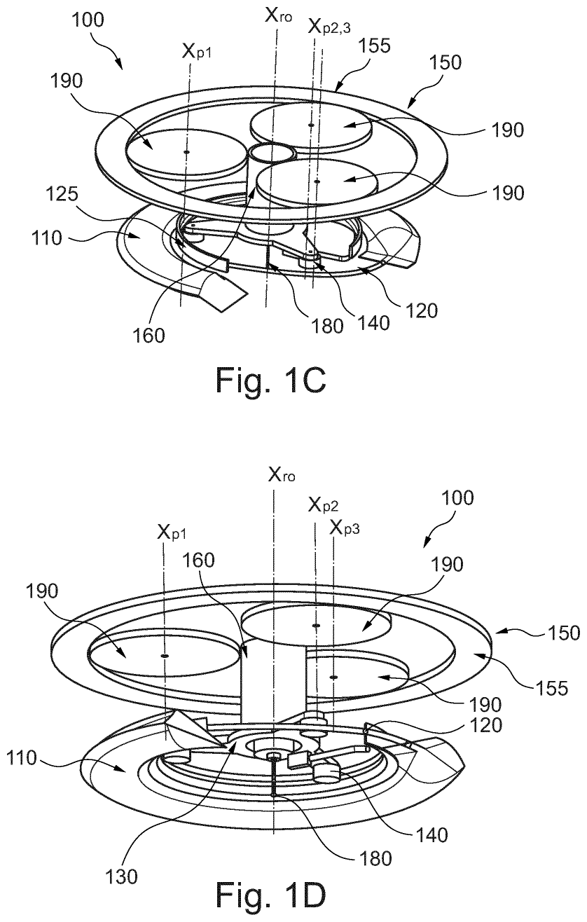

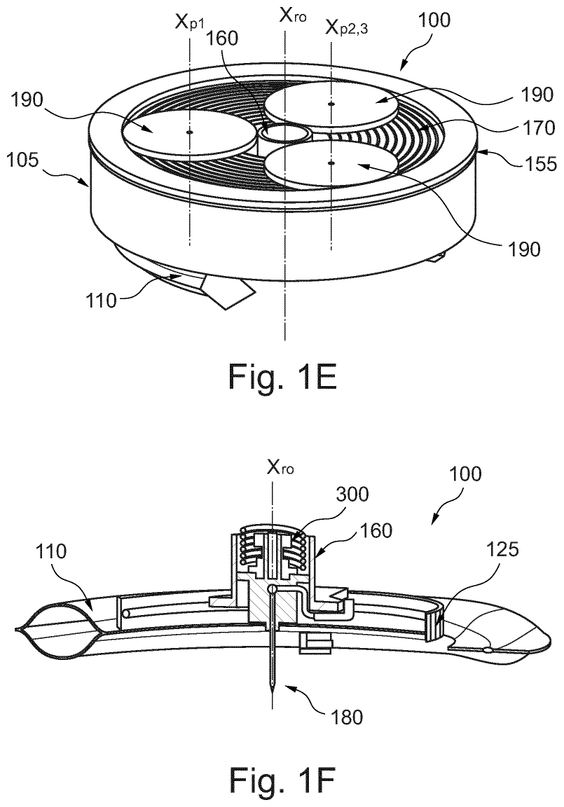

FIG. 1A shows a top view of a first embodiment of the current disclosure. It shows a reduced peristaltic pump arrangement 100. A squeeze unit 130 is arranged rotatable around a rotation axis Xro. The squeeze unit 130 comprises three rollers 140. The squeeze unit 130 comprises three arms which extend radially outward. Each of the rollers 140 is arranged at the radially outward ending of one arm of the squeeze unit 130 in relation to the rotation axis Xro. The rollers 140 may rotate around the rotation axis Xro along with the squeeze unit 130. During this rotation the rollers also rotate around their own rotation axis in a direction opposite to the rotation axis Xro. The rollers 130 are distributed evenly circumferentially around the rotation axis Xro such that they are separated by an angle of approximately 120°. The rollers 130 can also be separated by different angles. The rollers 140 can rotate separately around their own rotation axis and in addition to the rotation of the squeeze unit 140 around the rotation axis Xro. The rollers 140 are configured to squeeze a tube 120 in the direction radially outward. The tube 120 comprises a fluid material, e.g. a liquid medicament or medicament formulation. The tube 120 is arranged around the rotation axis Xro in the radial distance from the rotation axis Xro so that the rollers 130 can squeeze the tube 120. The tube 120 comprises a cross-section of a circle segment. One ending of the tube 120 is in fluid communication with the needle 180. This ending is arranged on a position further to the injection position along the rotation axis Xro than the circle segment of the tube 120. Further, a section of the tube 120 connects this ending with the circle segment, such that the squeeze unit 130 does not interfere with this section of the tube 120 during its rotation.

The tube 120 is mechanically supported by a cylindrical wall 125, which is placed radially further outward than the tube 120, so that when the rollers 140 squeeze the tube 120, it does not move radially outward. As the three rollers 140 rotate, they compress the tube 120 against the wall 125. This forces the fluid to move through the tube 120 to a needle 180. The tube 120 then expands back to its original shape, drawing more drug from a flexible primary pack 110, such as a pouch 110, which is in fluid communication with the tube 120 through an opening 135. The pouch 110 has a larger diameter than the tube 120 and it can contain more volume than the tube 120. As soon as there is fluid from the tube 120 moved to the needle 180, the fluid from the collapsible pouch re-fills the tube 120 with fluid. This ensures a constant flow of fluid from the tube 120 to the needle 180.

FIG. 1B shows an angled view of a reduced peristaltic pump arrangement 100 as shown in FIG. 1A. It shows an open ended cylindrical wall 125 for mechanically supporting the tube 120 when it is squeezed by the rollers 140. Further it is shown a needle 180 which is connected to a needle drive mechanism 300. The needle drive mechanism 300 is arranged inside a central wheel 160 of a planetary gear 150 (not shown here) and connected to or integrated with a housing 105. The needle drive mechanism 300 is mechanically decoupled from the central wheel 110, such that when the central wheel 160 rotates around the rotation axis Xro, the needle drive mechanism does not rotate.

FIG. 1C shows an angled top-view of the peristaltic pump arrangement, including a planetary gear 150 for driving the squeeze unit 130. The planetary gear has a central axis which is the rotation axis Xro. The planetary gear 150 comprises a ring wheel 155 which rotates around the rotation axis Xro. The planetary gear has further three planetary wheels 190 which are in rotational communication with the ring wheel 155 and a central wheel 160, which rotates around the rotation axis Xro. The planetary wheels 190 rotate around the central wheel 160 and at the same time they rotate around their own rotation axis Xp1, Xp2, Xp3. The planetary wheels 190 each have the same diameter which is larger than the diameter of the central wheel 160 and smaller than the diameter of the ring wheel 155. Each of the wheels ring wheel 155, planetary wheels 190 and central wheel 160 comprise gear wheels so that their rotation is interlinked, so that a gear ratio is established which determines the force required to turn the radially outermost ring wheel 155 and the force which is required to turn the central wheel 160. The central wheel 160 is mechanically connected to the squeeze unit 130 such that a movement of the central wheel 160 causes a movement of the squeeze unit 130 in the same direction.

FIG. 1D shows an angled bottom-view of the peristaltic pump arrangement, including a planetary gear 150 for driving the squeeze unit 130 from a different angle compared to the view of FIG. 10.

FIG. 1E shows an angled view of the planetary gear 150 with a motor spring 170, e.g. a torsion spring. The planetary gear 150 is driven by the motor spring 170, thereby driving the ring wheel 155. The rotation of the ring wheel 155 is then translated with a predetermined gear ratio via the planetary wheels 190 to the central wheel 160.

In an example, a dead volume within the tube 120 can be minimised by using a small diameter tube 120. The diameter of the tube 120 can be for example 0.5 mm-1 mm, in particular 0.86 mm or 0.9 mm. In this example the motor spring 170 can only provide around 20-25 rotations when it is fully biased, but the pump requires 120 rotations to empty the primary pack. This necessitates the use of a gearing system to achieve the required number of pump rotations. For this reason a planetary gear with a gear ratio of 1:5 might be employed. This can be achieved by using epicyclical gears.

In this configuration there is no load applied to the pouch 110, as all movement of the fluid is done with pull and push through the operating reservoir. The pump-arrangement is, in principle, also suitable for patch pump applications.

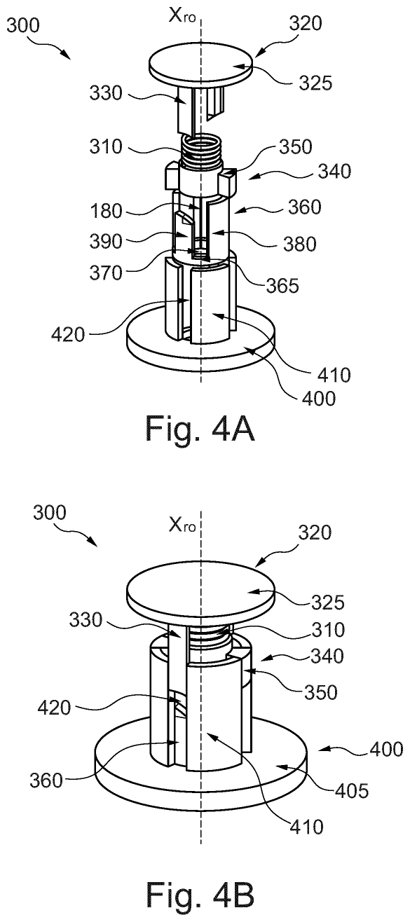

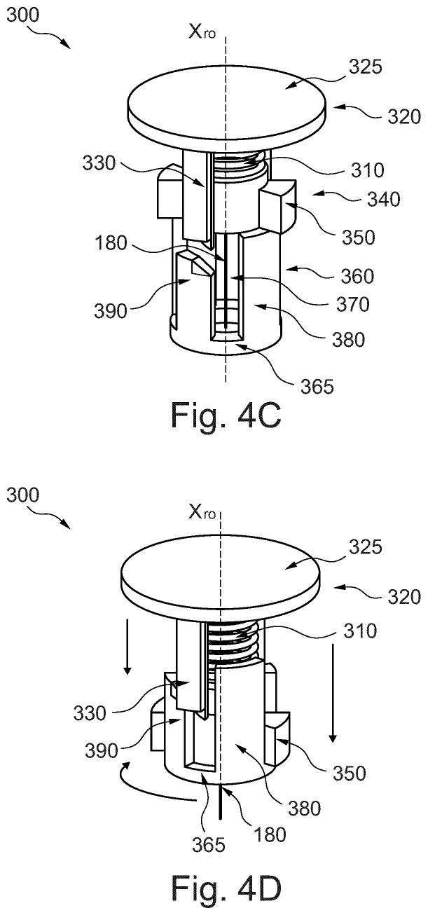

FIG. 1F shows cross-section of the peristaltic pump arrangement 100 and a needle drive mechanism 300, which comprises the needle 180 needle drive mechanism 300. The needle drive mechanism 300 is described in detail in FIGS. 4A-4D.

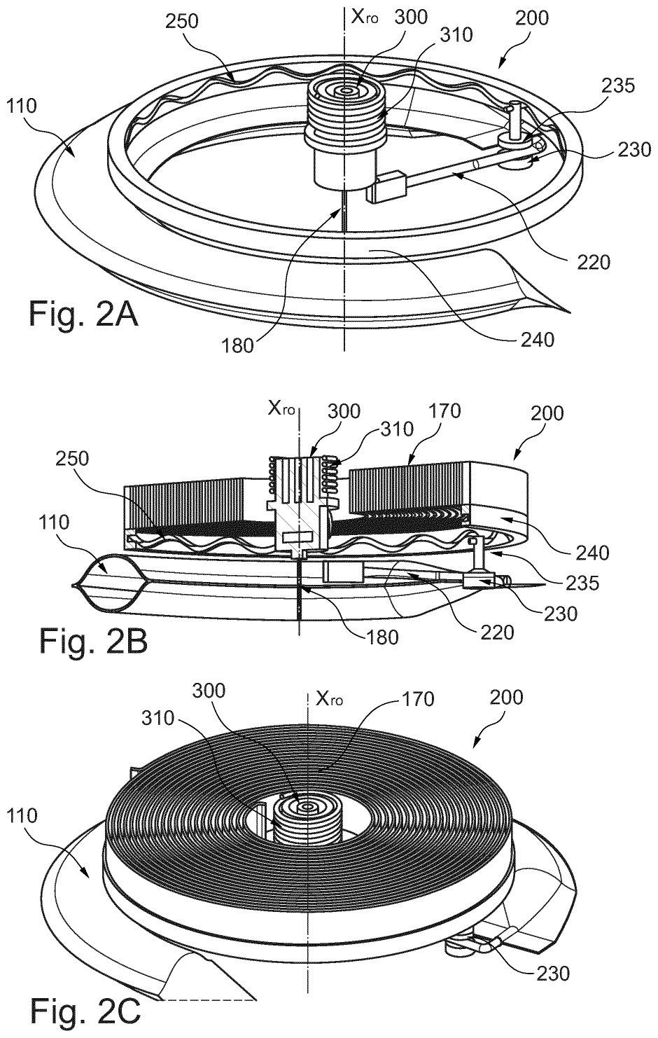

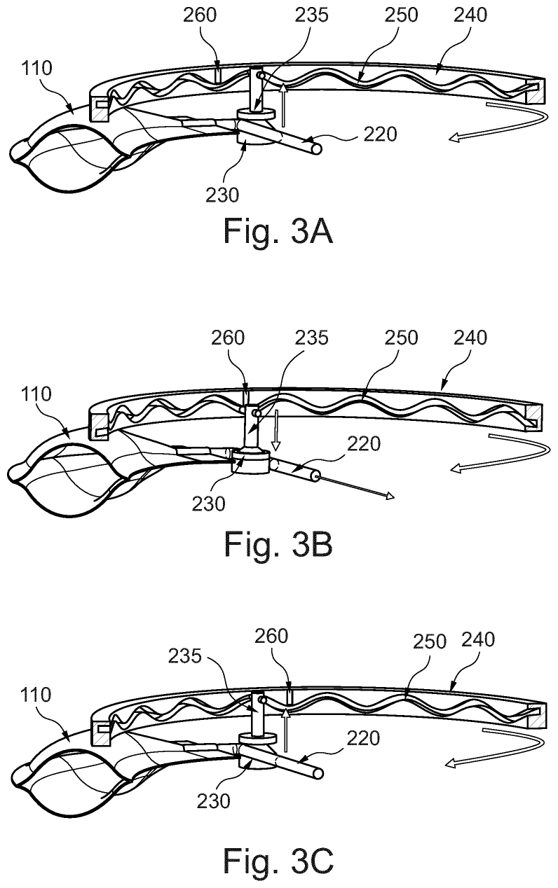

FIG. 2A shows a second embodiment of the current disclosure. It shows an angled view of the diaphragm pump arrangement 200. The diaphragm pump arrangement 200 comprises a diaphragm pump 230 with an diaphragm actuator 235 for actuating the diaphragm inside the pump in a direction along the rotation axis Xro. The diaphragm pump 230 is in fluid communication with a pipe 220 which comprises fluid, so that when the diaphragm actuator 235 is moved along the rotation axis Xro fluid is either pulled into the pump 230 or pushed out of the pump 230, depending on the direction in which the diaphragm actuator 235 is moving. Therefore, the movement of the diaphragm actuator 235 is not along the axis of movement of the liquid, but with an angle to it. The angle may be 90°, so that when the movement of the diaphragm actuator 235 is along the rotation axis Xro the fluid is moved perpendicular thereto. In order to enable a flow of fluid the diaphragm actuator 235 needs to move alternate in one direction along the rotation axis Xro followed by a movement in the opposite direction along the rotation axis Xro. The movement in one direction then may lead to fluid being drained into the diaphragm pump 230 from one side of the pipe 220, whereas the movement in the opposite direction thereafter, leads to fluid being pushed out of the diaphragm pump 230 on another side, the diaphragm pump 230 being arranged between the two sides. The pipe 220 is in fluid communication with the needle 180. The fluid which is pumped through the pipe 220 is the moved through the needle 180 for a dispensing operation. In this configuration the diaphragm pump 230 is stationary, but inside the diaphragm pump 230 the diaphragm actuator 235 moves along the rotation axis Xro. The movement of the diaphragm actuator 235 is caused by a cylindrical cam 240 which rotates around the rotation axis Xro. The cylindrical cam 240 comprises on its radial inside a sinusoidal shaped groove 250. The diaphragm actuator 235 is mechanically connected to this groove 250. When the cylindrical cam 240 rotates the diaphragm actuator 240 follows in its movement the shape of the groove 250. This movement is described in detail in FIGS. 3A-3C. The arrangement of the diaphragm actuator 235 being mechanically connected to the groove 250 and following its shape, leads to a translation of the amplitude of the sinusoidal shape to the diaphragm actuator 235. The diaphragm actuator 235 is thereby forced to an oscillating movement which is periodically, which ensures a continuous flow of fluid through the pipe 220 and through the needle 180. The arrangement further comprises a flexible primary pack, which may be a pouch 110 which is in fluid communication with the pipe 220 and which ensures that there is continuously fluid in the pipe 220 which can be moved to the needle 180.

The diaphragm pump arrangement comprises further a motor spring 170 (not shown here), which is mechanically connected to the cylindrical cam 240. The rotation of the cylindrical cam 240 is driven by the motor spring 240. When the motor spring 170 is biased it has a certain amount of potential energy. This energy is sufficient for a certain number of revolutions of the cylindrical cam 240 around the rotation axis Xro. The number of revolutions relates to the number of oscillations of diaphragm actuator 235, which in turn relates to an amount of fluid moved through the pipe 220 and finally through the needle 180. Therefore, by a set energy of the motor spring 170 the amount of fluid which is moved through the needle 180 can be pre-set. If the fluid comprises a medicament the medicament dose can be set by the energy of the motor spring 170.

To minimise the dead volume and maximise the dose accuracy, a low volume, high frequency pump is preferred. This can be achieved with a pump which has a pump stroke volume of the order of 1/50th or greater of the total volume delivered by the device. Experiments have shown that this requires more than 20 strokes (the motor spring provides 20 revolutions). By using a cylindrical cam with a sinusoidal wave profile, 420 pump strokes can be achieved without the need for additional gearing.

The arrangement further comprises a needle drive mechanism 300 with a drive spring 310. The needle drive mechanism 300 is described in detail in FIGS. 4A-4D.

FIG. 2B shows cross-section of the peristaltic pump arrangement 200 and the needle drive mechanism 300 with the drive spring 310. The needle 180 is arranged at the needle drive mechanism 300. The needle drive mechanism 300 is mechanically connected to the motor spring 170, such that if during the pumping process the needle 180 is filled with fluid a trigger (310) causes the needle to move along the rotational axis Xro into a tissue of a patient followed by an injection of the medicament once a predetermined depth of the needle into the patients tissue is reached. This is explained in more detail in FIGS. 4A to 4D.

FIG. 2C shows an angled view of the diaphragm pump arrangement 200 with the motor spring 170.

FIGS. 3A to 3C show a cut out of the diaphragm pump in different positions of operation. In order to illustrate this a marker 260 is indicated at the radial inside of the cylindrical cam 240, which shows the movement of the diaphragm actuator 235 caused by the cylindrical cam 240 and in relation to it. The cylindrical cam 240 in this example moves clockwise.

In FIG. 3A the marker 260 has moved to the positive peak of the amplitude of the sinusoidal wave, whereas the marker 260 is arranged further anti-clockwise. With the movement to the positive peak of the sinusoidal wave, which corresponds to an upward movement of the diaphragm actuator 235, fluid is drawn from the operating reservoir 220 into the diaphragm pump 230.

In FIG. 3B the cylindrical cam 240 has rotated further clockwise, and the sinusoidal wave has caused the diaphragm actuator 235 to move in a downward direction, to the negative peak of the sinusoidal wave, such that fluid is pushed through the operating reservoir 220 towards the needle 180. As shown in FIG. 3B the diaphragm actuator 235 has reached the position of the marker 260.

In FIG. 3C the movement of diaphragm actuator 235 repeats the movement as described in FIG. 3A. The cylindrical cam 240 continues to rotate and the sinusoidal wave causes the diaphragm actuator 235 to move further anti-clockwise and departing from the marker 260. The diaphragm actuator 235 reaches the next positive peak of the sinusoidal wave and draws further fluid from the operating reservoir 220 into the diaphragm pump 230.

This arrangement ensures a periodically movement of the diaphragm actuator 235, providing a continuous flow of fluid through the operating reservoir 220 to the needle 180.

The arrangement would work with the same principle if the cylindrical cam 240 moves anti-clockwise.

The device may have a height between 10-40 mm, and in particular a height between 15-30 mm. The base of the device may have a diameter between 45-90 mm, and in particular a diameter between 50-70 mm. In particular the height of the device may be smaller by a factor of more than three compared to a typical autoinjector comprising a syringe. This is advantageous for a user like a patient, because the distance from the skin to the position where the device is triggered is much less.

FIG. 4A shows an exploded view of a needle drive mechanism 300. Both the first embodiment according to FIGS. 1A-1F which relate to a peristaltic pump and the second embodiment according to FIGS. 2A-3C which relate to a diaphragm pump may comprise a needle drive mechanism 300 according to FIGS. 4A-4D unless otherwise stated.

The needle drive mechanism 300 comprises a trigger button 320, a drive spring 310, a needle holder 340, a collar 360 and a base element 400. The needle 180 is mechanically connected to the needle holder 340.

The trigger button 320 comprises a trigger button main body 325 and two trigger button arms 330 extending from the trigger button main body 325 along the rotation axis Xro. The trigger button main body 325 comprises a cylindrical shape wherein the height or thickness is smaller than the diameter forming an overall disc-like shape. The trigger button main body 325 can also comprise any other shape such as a rectangular or squared thin plate. The trigger button main body 325 may be connected to or integrated with the housing 105 of the device and then may have the same thickness as the housing 105. The two trigger button arms 330 are arranged opposite to each other with respect to the rotation axis Xro. Each of the trigger button arms 330 comprises a radial outward part and a radial inward part. Each of the trigger button arms 330 comprises an oblique surface on the ending away from the trigger main body 325 with respect to the rotation axis Xro. The oblique surfaces are arranged on the radial inward part.

The drive spring 310 is arranged between the trigger button main body 325 and the needle holder 340 along the rotation axis Xro and is expandable and compressible along the rotation axis Xro. The collar 360 can be arranged inside the base element 400. When the drive spring 310 is compressed the needle holder 340 is arranged between the collar 360 and the drive spring 310.

The needle holder 340 comprises a main body with a cylindrical shape and two needle holder ledges 350 which extend radially outward from the main body and are arranged on opposite sides with respect to the rotation axis Xro. The needle holder 340 is operatively connected to the drive spring 310, such that when the drive spring 310 expands along the rotation axis Xro the needle holder 340 and the needle 180 move along the rotation axis Xro away from the trigger button main body 325. The needle holder ledges 350 can have the shape of a wedge, wherein their cross-section decreases radially inwardly.

The collar 360 comprises a collar base 365, two collar holding arms 380 and two collar trigger arms 390. The two collar holding arms 380 and the two collar trigger arms 390 each extend from the collar base 365 towards the trigger button main body 325 along the rotation axis Xro. The two collar holding arms 380 are arranged on opposite sides with respect to the rotation axis Xro. The two collar trigger arms 380 are arranged on opposite sides with respect to the rotation axis Xro. The collar holding arms 380 and the collar trigger arms 390 are arranged alternating around the rotation axis Xro and are spaced apart by collar spacings 370. The collar holding arms 380 comprise a rectangular shape. The collar trigger arms 390 also comprise a rectangular basis-shape but with an oblique surface at their endings directing away from the collar base 365 and facing the oblique surfaces of the radial inward part of the trigger button arms 330. It is also possible that wither the trigger button arms 330 or the collar trigger arms 390 comprise oblique surfaces. For example when the only trigger button arms 325 comprise oblique surfaces, a movement along the rotation axis Xro would still lead to a rotation of the collar 360 when the oblique surface pushes on an edge of a collar trigger arm and thereby further moving along the rotation axis Xro. The collar base 365 comprises a hole such that the needle 180 can move through the hole along the rotation axis Xro.

The base element 400 comprises a base element main body 405 and four base element arms 410 which are each extending from the base element main body 405 in the direction towards the trigger button main body 325 along the rotation axis Xro. The base element main body 405 comprises a cylindrical shape wherein the height or thickness is smaller than the diameter forming an overall disc-like shape. The base element main body 405 can also comprise any other shape such as a rectangular or squared thin plate. The base element main body 405 may be connected to or integrated in the housing 105 of the device. The base element arms 410 extend from the base element main body 405 along the rotation axis Xro and are arranged around the rotation axis Xro, thereby enclosing a volume, which is configured to receive the collar 360 when it the needle drive mechanism 300 is assembled. The base element arms 410 are separated towards each other by spacings 420. Two of the spacings 420 are configured to receive the radial outward part of the trigger button arms 330 such that the trigger button arms 330 are axially guided within the spacings 420 but secured against rotation. The other two spacings are configured to received the needle holder ledges 350 along the rotation axis Xro which are then secured against rotation. The base element main body 405 comprises a hole such that the needle 180 can move through the hole along the rotation axis Xro to the outside of the device for injection. The hole can be sealed with a sealing, which can be penetrated by the needle.

FIG. 4B shows a view of the needle drive mechanism 300 in an assembled state. The drive spring 310 is compressed between the trigger button 320 and the needle holder 340 with respect to the rotation axis Xro, so that the drive spring 310 biases the needle holder 340. The trigger button arms 330 have entered the base element spacings 420 partially so that they can move along the rotation axis Xro wherein they are guided by the base element arms 410 which are forming the base element spacings 420. When the trigger button arms 330 are axially movable inside the base element spacings they are secured against rotation by the base element arms 410.

Alternatively the trigger button arms 330 could be also secured against rotation by the needle holder 340. For the cylindrical body of the needle holder 340 may comprise an additional groove which is oriented along the rotation axis Xro, and which is configured to mechanically guide the trigger button arms 330 along the rotation axis Xro, thereby securing them against rotation.

The needle holder ledges 350 are in the same way arranged in base element spacings 420. In this way the needle holder ledges 350 are secured against rotation by the base element 400 and if the base element 400 is a part of the housing 105 of the device the needle holder ledges 350 and the needle holder 340 are secured against rotation by the housing 105. Further the needle holder ledges 350 are axially blocked by the collar 360. The collar 360 is arranged inside the space which is enclosed by the base element arms 410.

The needle holder 340 comprises a hole for receiving a pipe (not shown). The hole can be directed radially inwardly from the needle holder ledge 350. The pipe is in fluid communication with the needle 180 and either the tube 120 according to the embodiment of FIGS. 1A-1F or the pipe 220 according to the embodiment of FIG. 2A. In this way the fluid of the pouch 110 can be transferred to the needle 180 for dispension. In the embodiment of the peristaltic pump according to FIGS. 1A-1F, a part of the pipe is arranged between the base element 400 and the central wheel 160 where it is connected to the tube 120, so that one of the endings of the pipe can be connected to the tube 120 for establishing a fluid communication. This arrangement ensures that the pipe is de-coupled from the rotational movement of the central wheel 160.

FIG. 4C shows a view of the assembled needle drive mechanism 300 without the base element 400 where the drive spring 310 is compressed between the trigger button 320 and the needle holder 340 with respect to the rotation axis Xro, so that the drive spring 310 biases the needle holder 340. The needle holder 340 is arranged between the drive spring 310 and the collar 360 with respect to the rotation axis Xro. The needle holder ledges 350 are in mechanical contact with the ending surfaces of the collar holding arms 380, such that the force of the compressed drive spring 310 acts on the collar holding arms 380 through the needle holder ledges 350. The trigger button arms 330 are arranged radially outward with respect to the drive spring 310 and the needle holder 340. The endings of the trigger button arms 330 are facing the endings of the collar trigger arms 390 but are spaced apart axially. Both the endings of the trigger button arms 330 and the endings of the collar trigger arms 390 comprise an oblique surface, such that when the oblique surface of a trigger button arm 330 is pressed towards the surface of a collar trigger arm 390, the surfaces slide towards each other, and the additional movement along the rotation axis Xro by the trigger button arms 330, which applies a force to the collar trigger arms 390, causes the collar trigger arms 390 and the collar 360 to rotate.

FIG. 4D shows a view of the assembled needle drive mechanism 300 without the base element in which the drive spring 310 is expanded along the rotation axis Xro. The needle holder 340 has been moved along the rotation axis Xro in a direction away from the trigger button main body 325 due to the force of the expanding drive spring 310. Thereby, the needle holder ledges 350 have engaged into the collar spacings 370, thereby guiding the movement of the needle holder 340 along the rotation axis Xro.

When the needle drive mechanism 300 is in the status as shown in FIG. 4B or 4C, i.e. when the drive spring 310 is compressed and the needle holder 340 is blocked from moving in the direction away from the trigger button main body 325, and the trigger button 320 is pressed in a direction towards the drive spring 310 along the rotation axis Xro the trigger button arms 330 are moved towards the collar trigger arms 390. When the oblique surfaces of the trigger button arms 330 are pressed towards the surfaces of the collar trigger arm 390, the axial movement of the trigger button arms 330 cause a rotational movement of the collar trigger arms 390 and the collar 360. The rotation of the collar 360 is relative to the needle holder ledge 350. Because of the rotation of the collar 360 also the collar holding arms 380 rotate relative to the needle holder 340. During this rotation the needle holder ledges 350 are biased in the axial direction towards the collar holding arms 380 by the drive spring 310. The rotation of the collar holding arms 380 continues until at the collar spacings 370 face the needle holder ledges 350. At this point the needle holder ledges 350 are no longer blocked by the collar holding arms 380 and the trigger button arms 330 have disengaged from the collar trigger arms 390. The needle holder 340 then can axially move in a direction away from the trigger button main body 325. The collar holding arms 380 are longer than the collar trigger arms 390. It is also possible that the collar holding arms 380 are of the same length as the collar trigger arms 390. In this case the trigger button arms 330 needed to be adjusted in their length. The needle holder ledges 350 then engage in the collar spacings 370 and the needle holder 340 moves together with the needle 180 in a direction away from the trigger button main body 325 towards the base element 400 and further to the injection site under the force of the expanding drive spring 310.

The scope of protection is not limited to the examples given herein above. Any invention disclosed herein is embodied in each novel characteristic and each combination of characteristics, which particularly includes every combination of any features which are stated in the claims, even if this feature or this combination of features is not explicitly stated in the claims or in the examples.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir0); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N—(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C, CM-3, GLP-1 Eligen, ORMD-0901, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, TT-401, BHM-034. MOD-6030, CAM-2036, DA-15864, ARI-2651, ARI-2255, Exenatide-XTEN and Glucagon-Xten.

An examples of an oligonucleotide is, for example: mipomersen sodium (Kynamro®), a cholesterol-reducing antisense therapeutic for the treatment of familial hypercholesterolemia.

Examples of DPP4 inhibitors are Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

This patent application claims the priority of the European patent application 20315494.3, the disclosure content of which is hereby incorporated by reference.

### LIST OF REFERENCES

100 Peristaltic pump arrangement
105 Housing
110 Flexible primary pack
120 Tube
125 Cylindrical wall
130 Squeeze unit
135 Opening
140 Rollers
150 Planetary gear
155 Ring wheel
160 Central wheel
170 Motor spring
180 Needle
190 Planetary wheel
Xro Rotation axis
Xp1, Xp2, Xp3 Rotation axis of planetary wheels
200 Diaphragm pump arrangement
220 Pipe
230 Diaphragm pump
235 Diaphragm actuator
240 Cylindrical cam
250 Groove
260 Marker
300 Needle drive mechanism
310 Drive spring
320 Trigger button
325 Trigger button main body
330 Trigger button arm
340 Needle holder
350 Needle holder ledge
360 Collar
365 Collar base
370 Collar spacing
380 Collar holding arm
390 Collar trigger arm
400 Base element
405 Base element main body
410 Base element arm
420 Base element spacing

1. Autoinjector comprising
a housing (105) which is configured to receive an operating reservoir (120, 220) with a fluid,
a pump arrangement (100, 200), which is configured to drive the fluid from the operating reservoir (120, 220) towards and through an outlet (180) of the autoinjector in a dispensing operation.

2. Autoinjector according to claim 1, wherein the pump arrangement (100, 200) comprises a movable element (130, 230) which is operatively connected with the operating reservoir (120, 220) such that a movement of the movable element (130, 230) drives at least a portion of the fluid from the operating reservoir (120, 220) to the outlet (180).

3. Autoinjector according to any of the claim 1 or 2, comprising a reserve reservoir (110) which is in fluid communication with the operating reservoir (120, 220), and which is configured to supplement fluid to the operating reservoir (120, 220) during a dispensing operation.

4. Autoinjector according to any of the claims 1-3, comprising a circumferentially disposed element (125, 240) which encloses a rotation axis (Xro), and which is arranged adjacent to the reserve reservoir (110) radially inward.

5. Autoinjector according to any of the preceding claims, comprising a motor spring (170) which is configured to move the movable element (130, 230).

6. Autoinjector according to any of the preceding claims 2-5, wherein the movable element (130, 230) comprises a squeeze unit (130) with
at least one squeeze element (140) which is configured to squeeze the operating reservoir (120), wherein
the squeeze unit (130) is rotatable around the rotation axis (Xro), such that the at least one squeeze element (140) rotates around the rotation axis (Xro) and thereby squeezes the operating reservoir (120) causing the fluid to be driven within the operating reservoir (120).

7. Autoinjector according to claim 6, wherein the circumferentially disposed element (125) is radially arranged between the operating reservoir (120) and the reserve reservoir (110), such that it mechanically supports the operating reservoir (120) while it is squeezed by the at least one squeeze element (140) in radial direction.

8. Autoinjector according to any of the claim 6 or 7, comprising a rotary gear system (150) which is mechanically connected to the squeeze unit (130), and which is configured to cause the squeeze unit (130) to rotate around the rotation axis (Xro), wherein the speed of rotation of the squeeze unit (130) is determined by a gear ratio of the rotary gear system (150).

9. Autoinjector according to claim 8, wherein the motor spring (170) is mechanically connected to the rotary gear system (150) and provides rotation energy to the rotary gear system (150).

10. Autoinjector according to any of the claims 2-5, wherein the movable element (230) comprises a diaphragm actuator (235), which is integrated into a diaphragm pump (200) which is in fluid communication with the operating reservoir (220), wherein the diaphragm actuator (235) is configured to move parallel to the rotation axis (Xro) such that the movement causes at least a portion of the fluid to flow from the operating reservoir (200) through the diaphragm pump (230) to the outlet (180).

11. Autoinjector according to claim 10, wherein the circumferentially disposed element (240) is rotatable around the rotation axis (Xro) and comprises a mechanical guiding feature (250), wherein the diaphragm actuator (235) is connected to the mechanical guiding feature (250) such that when the circumferentially disposed element (240) rotates around the rotation axis (Xro) the diaphragm actuator (235) is moved parallel to the rotation axis (Xro), thereby causing the fluid to flow towards the needle (180).

12. Autoinjector according to any of the preceding claims, comprising an outlet drive mechanism (300) comprising
an outlet (180),
an interface element (340) which is connected to or integrated with the outlet (180), wherein the interface element (340) is movable from a first axial position to a second axial position along the rotation axis (Xro),
a trigger (320) which is operatively connected to the interface element (350) wherein
the trigger (320) is movable from a first trigger position to a second trigger position along the rotation axis (Xro), wherein
in the first trigger position the interface element (340) is releasably locked from moving from the first axial position to the second axial position, and
in the second trigger position, the interface element (340) is movable to the second axial position, wherein
the movement of the trigger (320) from the first trigger position to the second trigger position causes the interface element (340) to be released from the first axial position such that the interface element (340) is movable to the second axial position.

13. Autoinjector according to claim 12, comprising a holding element (360) which is in mechanical contact with the interface element (340), wherein
the holding element (360) is rotatable around the rotation axis (Xro) relative to the interface element (340) from a blocking position to a release position, wherein
in the blocking position the interface element (340) is releasably locked by the holding element (360) to move from the first axial position to the second axial position, and
in the release position the interface element (340) is movable from the first axial position to the second axial position, wherein
the movement of the trigger (320) from the first trigger position to the second trigger position causes the holding element (360) to rotate from the blocking position to the release position.

14. Autoinjector according to claim 12 or 13, comprising an outlet drive unit (310) which is operatively connected to the trigger (320) and to the interface element (340), wherein
the outlet drive unit (310) being configured to provide energy for moving the interface element (340) from the first axial position to the second axial position, wherein the outlet drive unit (310) has a first drive unit state and a second drive unit state, wherein
in the first drive unit state the outlet drive unit (310) has energy stored and the interface element (340) is in the first axial position and the holding element (360) is in the blocking position and the interface element (340) is prevented from moving to the second axial position, and
in the second drive unit state the outlet drive unit (310) can transfer energy to the interface element (340) so that the interface element (340) is moved the first axial position to the second axial position along the rotation axis (Xro) when the holding element (360) is in the release position, wherein
the movement of the trigger (320) from the first trigger position to the second trigger position causes the outlet drive unit (310) to change from the first drive unit state to the second drive unit state.

15. Autoinjector, according to any of the preceding claims, being a disposable or single-use device, for providing a single dose.

16. Autoinjector, according to any of the preceding claims, wherein
the autoinjector comprises a reserve reservoir (110) which is in fluid communication with the operating reservoir (120, 220) and which is configured to supplement fluid to the operating reservoir (120, 220) during a dispensing operation,
the autoinjector comprises a circumferentially disposed element (125, 240) which encloses a rotation axis (Xro) and which is arranged adjacent to the reserve reservoir (110) radially inward,
wherein the circumferentially disposed element (125) is radially arranged between the operating reservoir (120) and the reserve reservoir (110) and mechanically supports the operating reservoir (120).

17. Autoinjector, according to claim 6 or any one of claims 7 to 16 in its dependency of claim 6, wherein
the squeeze unit (130) comprises an arm which is orientated in radial direction,
the squeeze element (140) is a roller arranged at one end of the arm,
the squeeze element (140) is configured to rotate around a further rotation axis while rotating at the same time around the rotation axis, wherein the further rotation axis is parallel to the rotation axis,
the squeeze unit (130) is arranged such that when the squeeze element (140) squeezes the operating reservoir (120), the squeeze element (140) presses against the operating reservoir (120) in radial outward direction.
